(19) United States
(12) Patent Application Publication
(10) Pub. No.: US 2006/0287699 A1
(43) Pub. Date: Dec. 21, 2006

(54) NOSE ICE PACK ASSEMBLY

(76) Inventor: STEVEN ADAM RIEDLE, South Bend, IN (US)

Correspondence Address: BOTKIN & HALL, LLP, 105 E. JEFFERSON BLVD., SUITE 400, SOUTH BEND, IN, 46601, US

(21) Appl. No.: 11/421,069

(22) Filed: May 31, 2006

Related U.S. Application Data
(60) Provisional application No. 60/687,601, filed on Jun. 6, 2005.

(51) Int. Cl. A61F 7/00

(52) U.S. Cl. 607/109; 607/114

(57) ABSTRACT

A nose ice pack is disclosed for promoting clotting to stop a bleeding nose. A flexible cover sheaths three adjacent coolant pockets, with two spaces between the pockets. A back flap is provided that is hinged over the back of the flexible cover to provide extra protection and space for pertinent information.

### CROSS REFERENCE TO RELATED APPLICATIONS

This application is a continuation of Provisional Application No. 60/687,601 filed May 27, 2006, and claims the benefit thereof.

### BACKGROUND OF THE INVENTION

The present invention relates generally to ice packs, and more particularly, to an ice pack assembly suitable for use on a human nose.

Various configurations of ice pack assemblies have been used as an effective method to promote clotting and stop a bleeding nose, whether the cause is due to injury, a blood-clotting disorder, or any other malady. Most notably, however, such ice pack configurations do not lend themselves to conform specifically around a human nose, allowing for a user to pinch the nose while applying the ice pack assembly.

### SUMMARY OF THE INVENTION

Accordingly, it is an object of the present invention to provide a nose ice pack assembly for use in promoting clotting to stop a bleeding nose.

It is another object of the present invention to provide a nose ice pack assembly that is flexible and capable of being pinched around a human nose.

It is a further object of the present invention to provide improved elements of a nose ice pack assembly for the purposes described which is inexpensive and fully effective in accomplishing its intended purposes.

In one embodiment, a flexible cover is placed over three adjacent pockets of coolant material, with two spaces between the pockets. When in use around a nose, the two outside coolant pockets are folded inwardly to rest against the inside coolant pocket. A separate flap is hinged over the back of the flexible cover to provide extra protection for the user's fingers while holding the nose ice pack assembly in place and to provide space for pertinent information, such as instructions, to be printed on the pack assembly.

The above and other objects and advantages of the present invention will become more apparent from the following detailed description when taken in conjunction with the accompanying drawings.

### BRIEF DESCRIPTION OF THE DRAWINGS

FIG. 1 is a front perspective view of anose ice pack assembly, according to the present invention;

FIG. 2 is a side elevational view of the nose ice pack assembly, the opposite side being a mirror image;

FIG. 3 is a side elevational view of the nose ice pack assembly, showing a back flap, the opposite side being a mirror image;

FIG. 4 is a front perspective view of the nose ice pack assembly, showing a back flap;

FIG. 5 is a front elevational view of the nose ice pack assembly, showing a back flap;

FIG. 6 is a back elevational view of the nose ice pack assembly, showing a back flap; and

FIG. 7 is a bottom plan view of the nose ice pack assembly, shown in use on a human nose.

### DETAILED DESCRIPTION OF INVENTION

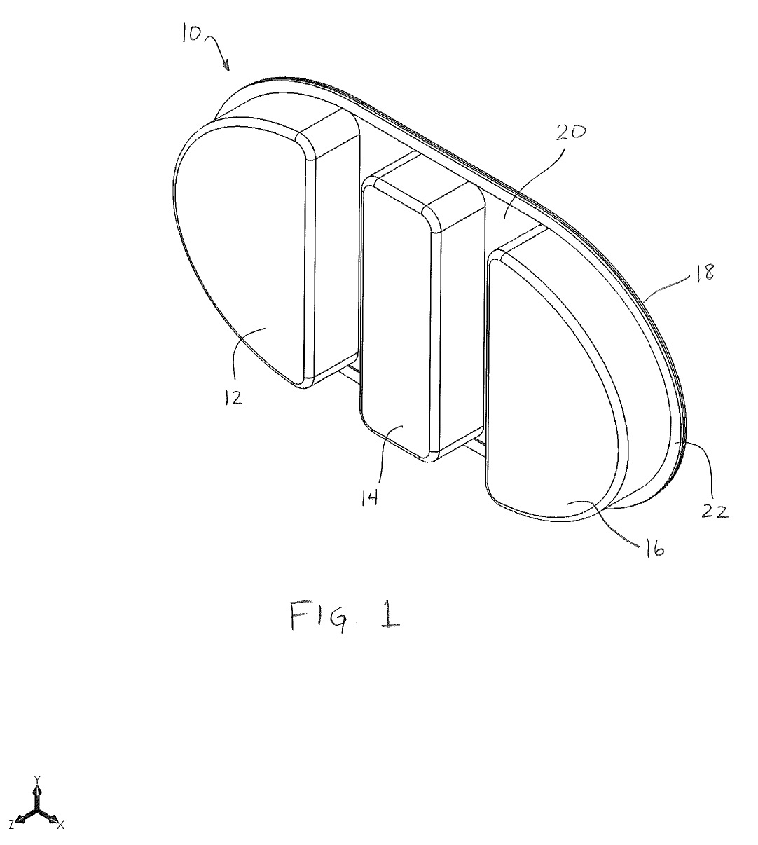

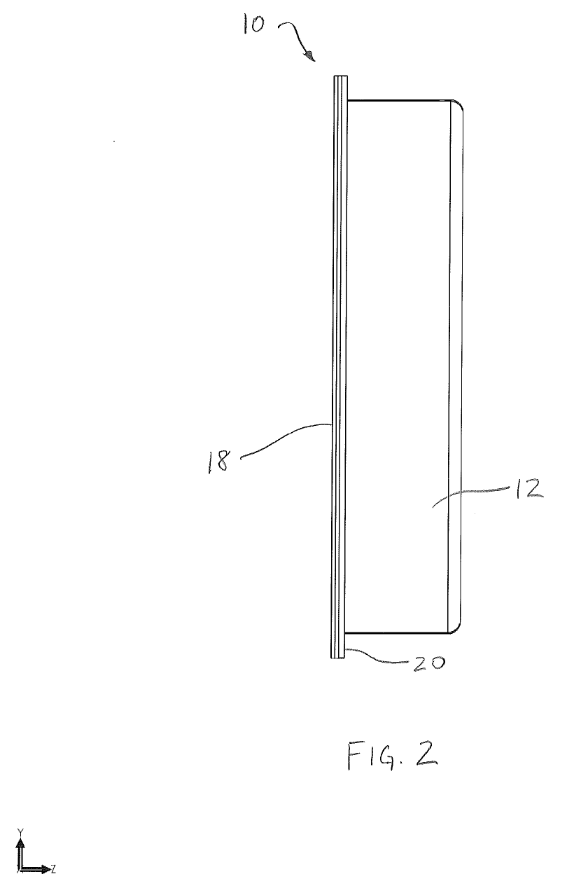

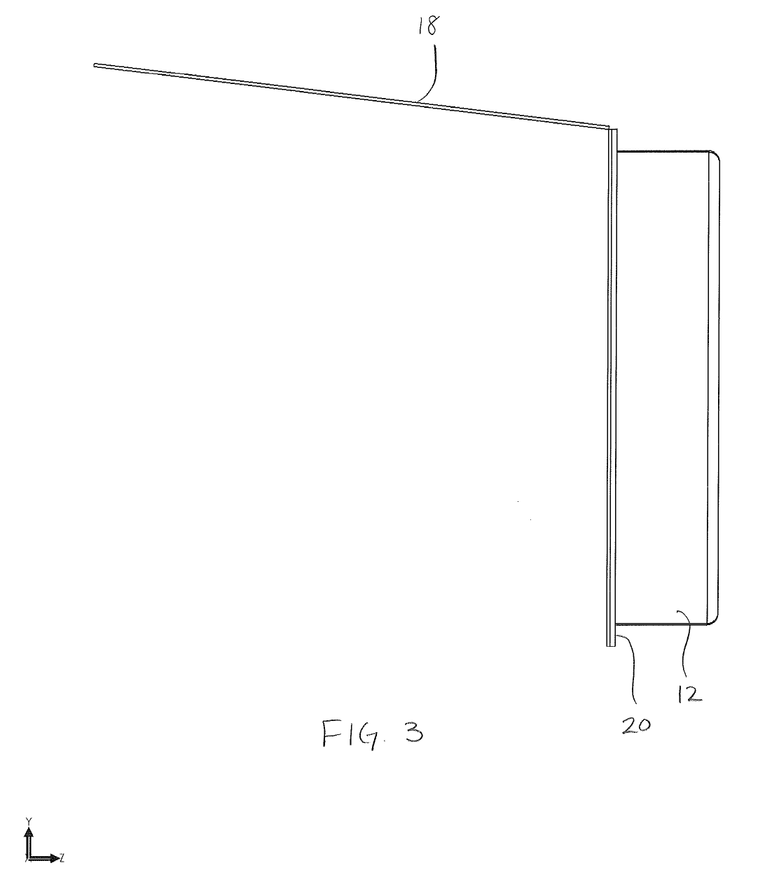

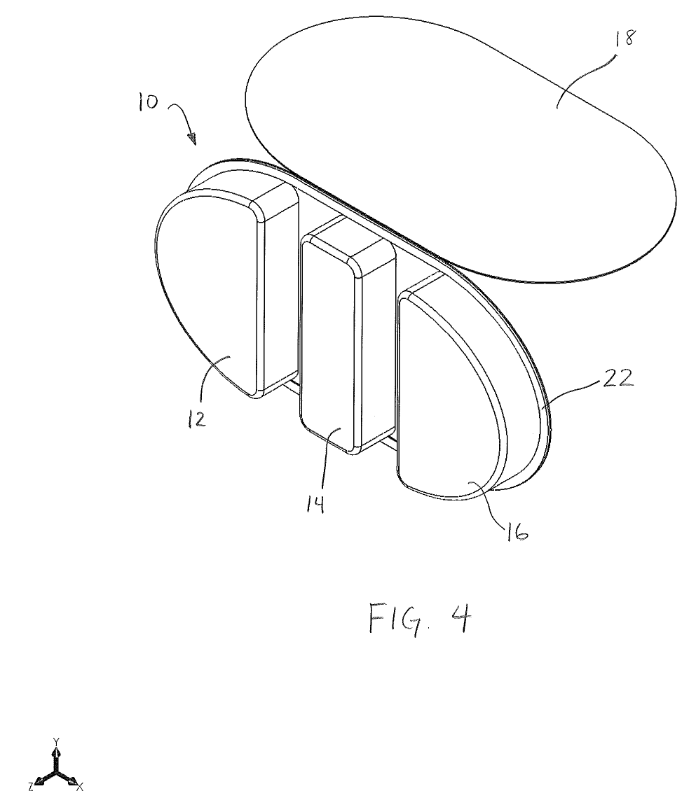

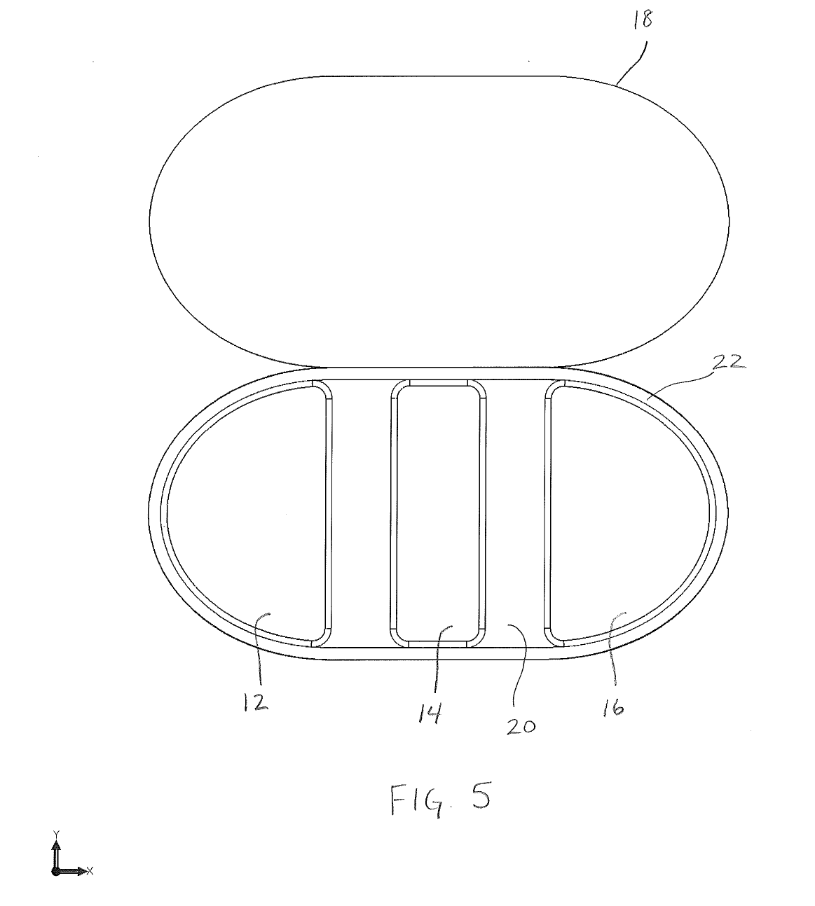

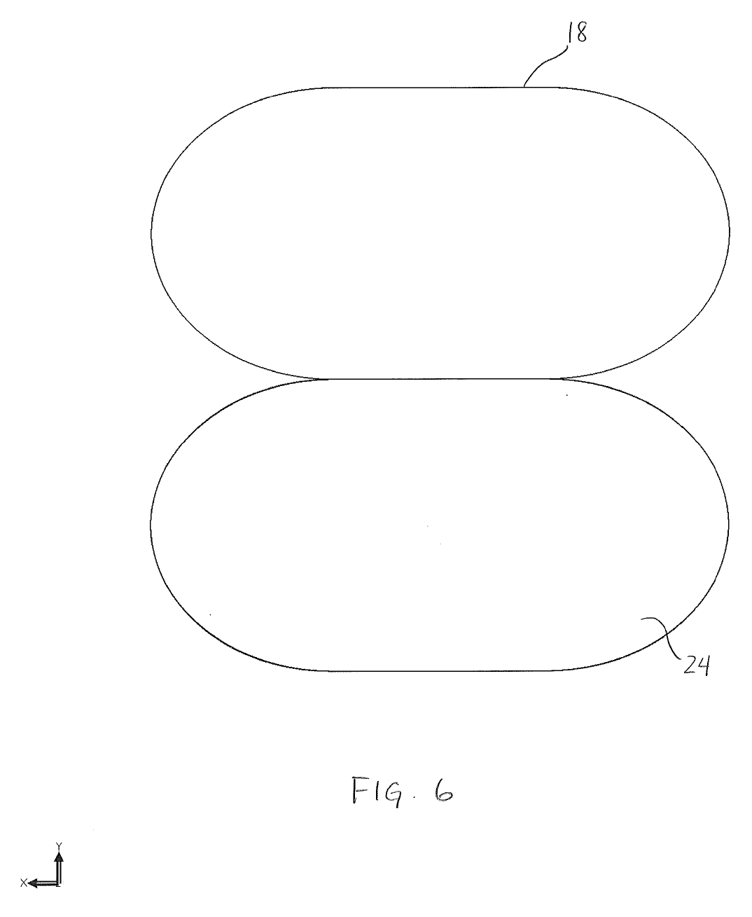

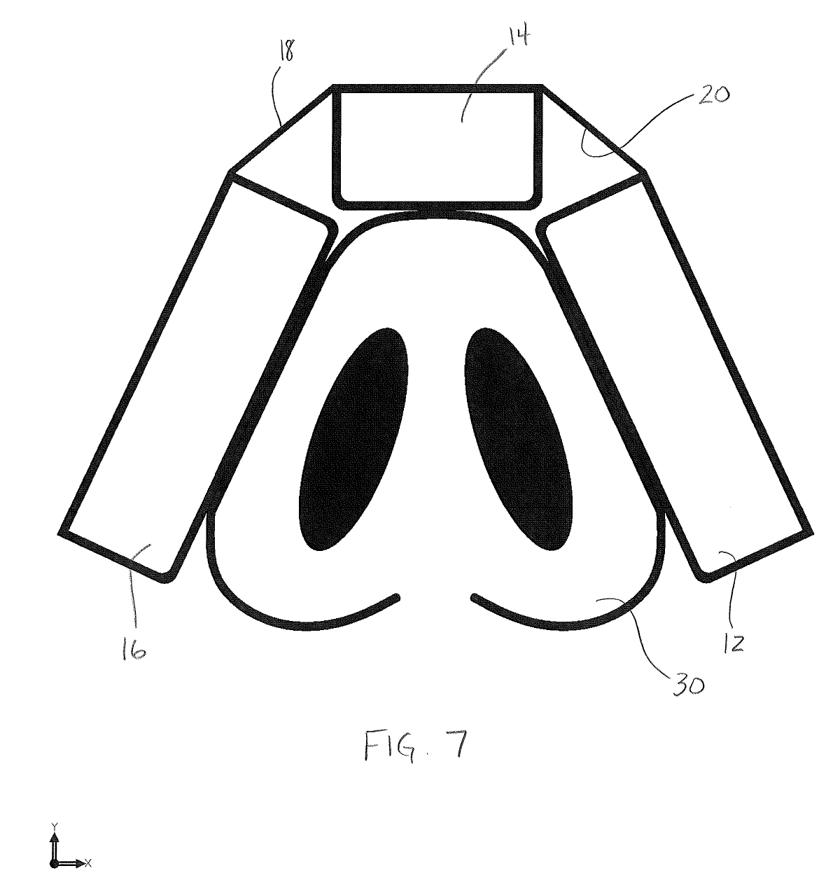

With reference to FIGS. 1-6, a nose ice pack assembly 10 is shown for use in preventing nose bleeds and promoting clotting. The nose ice pack assembly 10 may be constructed of various flexible materials to allow for the assembly 10 to be folded around a human nose 30, as shown in FIG. 7.

The nose ice pack assembly 10 is a single member containing three separate pockets in which coolant is dispersed: a first outer pocket 12, a second outer pocket 16 and an inner pocket 14. In one embodiment, an edge 22 is provided which extends beyond the pockets 12, 14, and 16. Two spaces, indicated collectively as 20, are provided to fold the pockets 12 and 16 inward when the nose ice pack assembly is in use on a human nose. The pockets 12 and 16 thus rest against pocket 14 in such use.

A separate flap 18 is hinged over the back 24 of the flexible material of the assembly 10 to provide extra protection for the fingers of a user of the assembly 10 while holding the assembly 10 in place and to provide space for pertinent information, such as instructions, to be printed on the back of the assembly.

The coolant pockets 12, 14, and 16 can be filled with a variety of coolant materials, including gel material or a chemical solution with an activator, allowing for instant cooling to take place.

It is to be understood that the present invention is not limited to the embodiments described above, but encompasses any and all embodiments within the scope of the following claims.

What is claimed is:

1. A nose ice pack assembly comprising:
a flexible cover;
first and second coolant pockets being formed from the flexible cover;
a third coolant pocket spaced between said first and second coolant pockets;
spaces of the flexible cover between the coolant pockets; and
a back flap formed from the flexible cover.

2. The nose ice pack assembly of claim 1 wherein the first and second coolant pockets are able to be folded inwardly; and

3. The nose ice pack assembly of claim 1, wherein said flexible cover is made of plastic.

4. The nose ice pack assembly of claim 1, wherein said coolant pockets are filled with a coolant gel.

5. The nose ice pack assembly of claim 1, wherein said coolant pockets are filled with a chemical solution with an activator allowing for instant cooling of the chemical solution.
